(19) United States
(12) Patent Application Publication
(10) Pub. No.: US 2004/0242454 A1
(43) Pub. Date: Dec. 2, 2004

(54) System and method for micro-dose, multiple drug therapy

(76) Inventor: Stephen I. Gallant, Cambridge, MA (US)

Correspondence Address: Robert M. Asher, Bromberg & Sunstein LLP, 125 Summer Street, Boston, MA, 02110-1618, US

(21) Appl. No.: 10/452,705

(22) Filed: Jun. 2, 2003

(51) Int. Cl. A61K 31/00; G06F 17/60; G06F 19/00; G01N 33/48; G01N 33/50

(52) U.S. Cl. 514/1; 702/19; 705/3

(57) ABSTRACT

A disclosed method of developing a drug comprises, in one embodiment: selecting a sub-group of constituent drugs from a potential group, wherein the selection is made on the basis that an expected effectiveness of a combination drug comprising all of the compounds of the selected sub-group exceeds a pre-determined minimum threshold measure of expected effectiveness against a target of the drug, and such that a side-effect measure for each expected side-effect of the combination drug is less than a corresponding pre-determined maximum threshold measure of side-effect tolerability for each side-effect; and selecting a dosage for each drug of the sub-group such that each dosage is less than a dosage that would be used as a fully effective dosage of the drug when used individually against the target of the drug. Related methods of treating patients, combination drug formulations, and computer-implementations of the methods are also disclosed.

The present application claims priority from U.S. Provisional Application, Ser. No. 60/385,030 filed May 31, 2002, which is incorporated herein by reference.

### TECHNICAL FIELD

The invention relates to systems and methods for drug discovery and therapy, and to the fields of combinatorial chemistry and machine learning.

### BACKGROUND ART

Efforts to develop a drug typically involve a great deal of time and expense, and risk failing because the drug is ineffective or has serious side effects. Presently, drug discovery usually involves a deep understanding of underlying biological and chemical systems, a great deal of experimentation, statistical modeling, and drug trials on animals and humans.

Some drug therapies, such as HIV therapies, involve combinations of several potent drugs in a drug “cocktail,” composed of a mixture of individually effective and tolerable drugs that is discovered by trial-and-error. However, such “cocktails” risk producing undesirable side effects from each of the components of the mixture.

### SUMMARY OF THE INVENTION

In one embodiment according to the invention, a method of developing a drug comprises: selecting, from a group of potential component drugs, a sub-group of component drugs, wherein the selection is made on the basis that an expected effectiveness of a combination drug comprising all of the component drugs of the selected sub-group exceeds a pre-determined minimum threshold measure of expected effectiveness against a target of the drug, and such that a side-effect measure for each expected side-effect of the combination drug is less than a corresponding pre-determined maximum threshold measure of side-effect tolerability for each side-effect; and selecting a dosage for each component drug of the sub-group such that each component dosage is less than a dosage that would be used as a fully effective dosage of the component drug when used individually against the target of the drug.

In related embodiments, the method may comprise obtaining, for each potential component drug, a measure of expected effectiveness against the target of the drug; and, for each potential component drug, a side-effect measure for each expected side-effect of the combination drug. Obtaining such measures may comprise using data from experiments already conducted by others, or estimating the measures using a predictive model. The measures may be adjusted based on the characteristics of an individual patient who will be receiving the combination drug, or of a group of patients that share a common characteristic. The measure of effectiveness against the target may comprise an expected probability of effectiveness, and the side-effect measure may comprise an expected probability of occurrence of an undesirable side effect. The measures may be obtained using functions that express the measure in terms of other variables, such as component dosages or patient characteristics. Such functions may reflect interactive effects between component drugs. Patient characteristics may be derived from laboratory tests or genetic information or from a gene chip array.

In a further, related embodiment, a sub-group of compounds for the drug formulation, and the quantity of each compound relative to all other compounds in the sub-group, is determined by performing an optimization of a combination of effectiveness measures and at least one combination of side-effect measures. Performing the optimization may include keeping one or more combinations of side-effect measures below a given maximum-threshold-side-effect measure for one or more side effects. Performing the optimization may also include using linear or non-linear programming.

In a further, related embodiment, selecting the sub-group of component drugs comprises initially selecting all component drugs from the group of potential component drugs that exceed a pre-determined minimum threshold measure of expected component effectiveness against the target; and then, for each side-effect measure of an expected side-effect of the combination drug that exceeds the corresponding pre-determined maximum threshold measure of side-effect tolerability, iteratively removing component drugs from the sub-group in decreasing order of an expected side-effect measure of each component drug, until the expected side-effect measure of the combination drug no longer exceeds the pre-determined maximum threshold measure of side-effect tolerability.

In another related embodiment, the method comprises setting constraints on the pre-determined maximum threshold measures of side effects to generate a feasible region of side-effects, and selecting the sub-group of component drugs in a manner that maximizes an objective function of expected effectiveness against the target, over the feasible region of side-effects. Setting the constraints may comprise requiring that the sum of the proportions of each component drug in the combination drug equals 1. Setting the constraints may also comprise varying the constraints based on sensitivity of an individual, or group of individuals, to at least one side-effect.

Another embodiment according to the invention comprises a method of treating a patient, the method comprising: developing a combination drug according to the above method of developing a drug; and administering the combination drug to a patient. Administering the drug to the patient may comprise administering several doses of the drug over a time interval.

In accordance with one embodiment of the invention, when the combination drug is administered in several doses over time, different combinations of component drugs may be used in different separately administered doses. In addition, delivery of different combinations of component drugs in different separately administered doses may be used in optimizing drug effectiveness with respect to the patient's system's response to the drug therapy over the time period of administration. In this regard, measures of effectiveness and side effects may be calculated using functions that take into account variables related to time-of-delivery of each component drug to the patient.

Another embodiment according to the invention comprises a combination drug formulated in accordance with the above method of developing a drug. A further related embodiment comprises a combination drug, so formulated, wherein each of the component drugs, if used at sufficient dosages to be expected to be effective against the target of the drug, would be expected to exceed a pre-determined maximum threshold measure of side-effect tolerability for at least one side-effect. Another further related embodiment comprises a combination drug, so formulated, used in combination with a partial or fully effective dosage of another drug, that is capable of being effective against the target, and of having tolerable side-effects, when used individually against the target.

Related embodiments of the invention implement the methods described in the form of a computer-program products.

### BRIEF DESCRIPTION OF THE DRAWINGS

The foregoing features of the invention will be more readily understood by reference to the following detailed description, taken with reference to the accompanying drawings, in which:

FIG. 1 shows an example of a table of data that is used in accordance with an embodiment of the invention as the basis for discovering a new drug therapy;

FIG. 2 is a block diagram of a process for developing a multiple drug therapy, in accordance with an embodiment of the invention; and

FIG. 3 is a block diagram of a solution method to the optimization of a non-linear predictive model to determine a sub-group of a drug formulation, in accordance with an embodiment of the invention.

### DETAILED DESCRIPTION OF SPECIFIC EMBODIMENTS

The fundamental problem in discovering a new drug therapy is finding a drug that produces a desired effect on a biological target, but also has acceptable side effects. Biological targets include conditions or states associated with a biological system or subsystem. Some examples of biological targets include diseases, blood pressure, pain associated with a part of the body, elasticity of tissue, water retention, and states such as alertness or shivering. Examples of biological systems and subsystems include humans, animals, plants, other types of organisms, the circulatory system of a human or animal, the heart of a human or animal, and a type of cell in an organism. A biological target may include more than one condition or state, or more than one biological system. For example, a biological target may be blood sugar level and weight in a group of persons with diabetes. In some embodiments of the invention described herein, the biological target refers to a disease. Desired effects refer to some preferred change in a biological target. Thus, some examples of a desired effect on a biological target are elimination of a disease from an organ, lowering of blood pressure, and inducing a change in the metabolism of an organism. Side effects refer to some change in a biological target that is not intended. In some embodiments of the invention described herein, side effects refer to changes in a biological target that are detrimental or undesired.

The drug formulations identified and created by embodiments of the present invention include a sub-group of compounds, where a compound is a distinct entity. Non-limiting examples of compounds include a particular pharmaceutical agent, a chemical, and a naturally-occurring substance. In some embodiments of the invention, the compounds represent existing drugs for producing a desired effect, but having side effects. The sub-group of compounds may be selected from a plurality of compounds that are candidate entities for a drug formulation. A sub-group of compounds includes one or more compounds.

Embodiments according to the present invention may allow the creation of a drug therapy at a lower cost, with a greater effect on a desired biological target, and with less severe side effects than are found with previous methods of drug discovery.

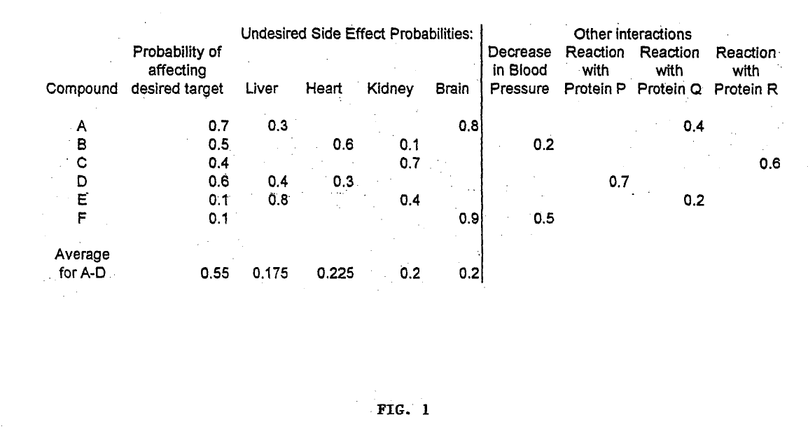

FIG. 1 shows an example of a table of data that is used in accordance with an embodiment of the invention as the basis for discovering a new drug therapy. The table contains a list of compounds in the first column, a list of probabilities that the compounds will affect a desired biological target in the second column, and lists of probabilities of undesirable side effects and other interactions (which may be effects that are neither desirable nor undesirable) in the remaining columns. The probabilities are expected values that may not necessarily reflect the true probability that a compound will have a particular affect.

In the example of FIG. 1, compound A is the most effective compound against the desired biological target (which may be, for example, a disease such as a pancreatic cancer), since it has the highest probability of affecting the biological target. However, it also has a high probability of producing brain and liver side effects, so that, taken individually, it is not a good candidate for use as an effective, tolerable drug. Each of the other listed compounds also has a high probability of producing undesirable side effects, when taken individually.

Therefore, in a method in accordance with an embodiment of the invention, a drug formulation is administered that is a mixture of a plurality of compounds, each of which is individually effective against a desired biological target, but with each compound being present in a small dose—which may be a micro-fraction of the dose of that compound that would be used if the compound was being used individually against the biological target as a drug. Such a method retains the high level of effectiveness against the desired biological target possessed by each of the compounds, but minimizes each of the undesirable side effects. As an illustrative example, consider a mixture of compounds A through D in FIG. 1, where each of A through D is a drug. Even using only four such compounds, each of which is individually effective against a desired biological target (with probabilities of affecting the desired biological target of 0.7, 0.5, 0.4, and 0.6), a mixture of the compounds retains a high overall effectiveness, while reducing the undesirable side effects. Assume, for example, that compounds A through D are mixed in even proportions, and that their effectiveness probabilities and side effect probabilities scale linearly with reduced dosages. Then the probability of the mixture of compounds affecting the desired biological target is the average of the individual compounds' probabilities of affecting the desired biological target, i.e. 0.55, an effective probability against the desired biological target. However, the probability of the mixture producing each undesirable side effect is low, as found by averaging each of the side effect probabilities to obtain probabilities of 0.175, 0.225, 0.2, and 0.2 for undesirable side effects against the liver, heart, kidney, and brain respectively.

In practice, an embodiment according to the invention may utilize many more than the four compounds used in the example, and use a micro-dose of each compound. As the number of compounds increases, the individual dosage amount of each compound is correspondingly made smaller. Since each of the compounds delivers some effect against the same biological target, the many compounds combine to have a strong efficacy against the biological target. However, because each compound has different side effects, and is used only in a small dose, individual side effect probabilities are reduced.

In addition to reducing undesirable side effects, such an approach according to an embodiment of the invention has a number of advantages.

First, a mixture of a large number of compounds may be more effective against a biological target. Each compound may attack different weaknesses in a biological target; or a large number of compounds may make it more difficult for an organism (such as the HIV virus) to mutate and become resistant to the mixture of compounds.

Next, not all drugs in the mixture are required to be effective against the biological target. Suppose, for example, that because of bad data or a lack of understanding, a compound that is believed to be effective against a biological target actually has zero effectiveness. In such a case, a mixture of a large number of compounds according to an embodiment of the invention is still effective against the biological target, because the average probability of effectiveness of a large number of compounds is still high even when one compound is zero. In fact, as many as one-quarter to three-quarters of the compounds could be ineffective against the biological target, and the average of a large number of compounds could still be effective against the biological target. The task of drug discovery may, therefore, be easier and less expensive, because precise information about the effectiveness of all of the compounds is not needed. The expense of drug discovery in an embodiment according to the invention may also be reduced because the method uses compounds that have already been discovered, and therefore does not require development of new compounds. In the case that each of the compounds has previously secured FDA approval, less expense should be required to secure FDA approval for the combination drug.

An analogous advantage of an embodiment according to the invention is that even if one or many of the compounds drugs in the mixture has undiscovered adverse side effects, these side effects may be reduced to low levels, because the compound is used in a small dosage when mixed with the other compounds. Such an undiscovered adverse side effect is reduced to a greater degree if a greater number of compounds is used with correspondingly smaller individual dosage amounts of each compound. The probability of adverse side effects from a mixture of many compounds according to the invention may, therefore, be reduced by comparison with the probability of side effects in a single drug, so that the risk of failure of the drug development effort is reduced.

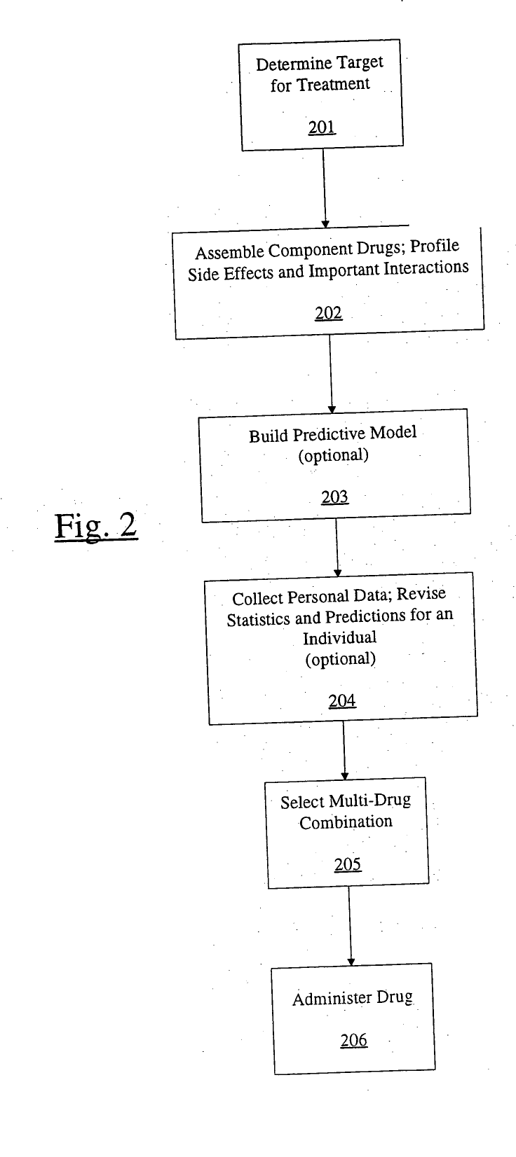

FIG. 2 is a block diagram of a method for developing a multiple drug therapy, in accordance with an embodiment of the invention.

In box 201, the embodiment of FIG. 2 involves determining a biological target for treatment, which may be, for example, a disease process in humans, animals, plants, or lower organisms; or a non-disease quality of the organism, such as its growth.

In box 202, the embodiment of FIG. 2 involves assembling a group of potential compounds for the multiple drug therapy, along with data on the compounds' side effects and important interactions. This data can be obtained from experiments that have already been conducted by others, for example in experimental databases or data libraries of activities of compounds; or can be estimated from predictive models commonly used for data mining. Examples of such predictive models include neural networks, Bayesian networks, decision trees, regression, support vector machines, and others known to those of skill in the art of data mining. An example of the result of the step of box 202 is a table of compound data, similar to that of the table shown in FIG. 1. However, it should be noted that measures of individual compound effectiveness and side effects need not necessarily be probabilities, as shown in FIG. 1. Other scores that reflect the expected activity of a compound may also be used. In accordance with one embodiment of the invention, the measures of effectiveness and side effects are obtained using functions or equations that compute expected ratings of effectiveness or side effects (such as a probability or other score) based on other variables (such as dosages or patient characteristics). Such functions may also take into account positive or negative interactive effects between compounds, since one compound may reduce or increase the effectiveness or side effects of another compound. Effectiveness and side-effect measures may be linear or non-linear functions.

In box 203 of the embodiment of FIG. 2, one or more predictive models may optionally be used to enhance the results and estimates of box 202. Similar predictive models to those described for box 202 may be used.

The predictive models utilized in boxes 202 and 203 need not be restricted to the art of data mining. Models may also take the form of an existing, predictive model that additionally incorporates data from experimental databases, data libraries, and other sources. The models may be linear, or utilize a more complicated combinatorial interaction between compounds.

In box 204 of the embodiment of FIG. 2, the results from previous steps may optionally be tailored to characteristics of an individual patient who will be receiving the multiple drug therapy, or of a group of patients that share a common characteristic (e.g. diabetics). Personal data about the individual (or group) is collected, and statistics and predictions are revised for the individual (or group). Data may be based upon a physical trait, e.g., a weakened heart, or a laboratory test administered to one or more patients. A test may be used to obtain genetic information which may be utilized. Genetic information may be obtained through any means known in the art, including the use of gene chip arrays.

Data concerning personal characteristics may be used to adjust existing figures in a predictive model to tailor the model for an individual or group. For example, an effectiveness measure of a drug combination or compound that is utilized in a model in box 202 or 203 may be adjusted according to a characteristic of a patient, or group of patients. This step may be omitted for producing a single drug that is given to a wide range of patients, but may be useful to enhance results for an individual or specialized group. The step may also be useful for an individual (or group of individuals) who has a particular sensitivity to one or more side effects, for example, a patient that has a weakened heart.

Next, in box 205, the embodiment of FIG. 2 involves selecting a sub-group of compounds from the plurality of compounds that is expected to be effective against the biological target while also having tolerable side effects; and selecting their dosages, both the total quantity of the combination and the quantity of each compound relative to every other compound in the sub-group. The relative quantity may be calculated on the basis mass, size, number, or other physical parameters of the compounds. The effectiveness of a sub-group in producing a desired effect on a biological target may be identified by a combination of effectiveness measures, which may be characterized by a probability, function, or other measure of expected activity as described in the discussion of box 202. Likewise, side effects may be identified by a combination of side-effect measures for each side effect; each combination of side-effect measures may also be characterized by a probability, function, or other measure of expected activity.

The predictive models in boxes 202 or 203 may be constructed to provide a method for calculating a particular combination of effectiveness measures, given a sub-group, a dosage of the sub-group, and relative quantities of the compounds in the sub-group. Likewise, the predictive models may provide a basis for calculating a combination of side-effect measures for a particular side effect, given a sub-group, a dosage of the sub-group, and relative quantities of the compounds in the sub-group. Each of the combination of measures may be derived from the individual compound measures for effectiveness and side effects. For example, a combination of effectiveness measures may be an average of the individual compound effectiveness measures in accordance with the quantities of each compound in the sub-group. Such averages may be based on size, weight, number and other physical parameters of the compounds. Other examples may account for interactions between compounds. Combinations of effectiveness measures or side-effect measures may be non-linear functions of the effectiveness measures or side-effect measures of the individual compounds of the sub-group; the effective measures and side-effect measures may also be functions, as well as taking any other form described earlier.

The specific compounds and relative quantities of each member of the sub-group may be chosen to optimize one or more combinations of side-effect measures associated with side effects. For example, the sub-group may be chosen to limit each of one or more side effects below a chosen maximum-threshold-side-effect measure. In addition, the choice of compounds and relative quantities may be chosen to optimize a combination of effectiveness measures of the sub-group. The optimizations may be combined together as well. One example of optimizing is to choose compounds and their quantities to maximize a combination of effectiveness measures, while maintaining one or more side-effect threshold measures below some predetermined levels. Another example of optimizing is to choose compounds and their relative quantities to achieve a desired minimum-threshold-effectiveness measure while maintaining one or more combinations of side-effect measures below threshold levels. Such an example may also include choosing one or more of the combinations of side-effect measures to minimize. In a third example of optimizing the compounds, and quantities thereof, are chosen such that a desired minimum-threshold-effectiveness measure is attained while incorporating as many compounds of the assembled group in box 202 as possible and maintaining side-effect measures below threshold levels. The latter technique diversifies the combination such that if a compound's actual effectiveness is below its expected effectiveness, as represented by the model, the presence of a variety of other compounds may insure that a desired level of effectiveness is maintained.

Note that in performing an optimization, an optimal point, defined by some type of global minimum or maximum, need not be achieved, though performing an optimization to achieve such an optimal point is a particular embodiment of the invention. Indeed, optimizing may include determining a sub-group that is remote from some optimal point in an absolute sense mathematically or physically (e.g. performing an optimization to determine a sub-group that has a combination of effectiveness measures above a desired level and one or more effectiveness measures below desired levels).

In one embodiment according to the invention, the combination of multiple drugs is selected by:

(1) selecting all compounds, from the table of potential drugs, that have a probability of effectiveness against the biological target (or other measure of effectiveness against the biological target) that exceeds a pre-determined threshold (for example, a probability of effectiveness of 0.4); and

(2) checking to see if the total expected adverse effect for a mixture of the selected compounds is greater than a certain threshold for any particular adverse effect. This may be performed, for example, by averaging the probabilities in each column of adverse side effects; or by taking a weighted average, when the proportion of each drug in the selected mixture is not equal. If the threshold is exceeded for any adverse effect column, then the compounds with the highest expected adverse effect in the given column are eliminated from the combination until all columns have an expected adverse effect below the threshold.

In another embodiment according to the invention, techniques of linear programming, or other combinatorial techniques (including non-linear programming, gradient descent, and genetic algorithms), may be used to select drugs, and their proportions (which need not necessarily be equal) in the multi-drug combination. In a particular embodiment, a function that describes the combination of effectiveness measures serves as an objective function which may be maximized, subject to constraints on a combination of side-effect measures for each side effect (e.g. keeping the combination below a given level). Other objective functions may also be formulated (e.g. minimizing one or more combinations of side-effect measures subject to the constraint of maintaining a combination of effectiveness measures above a minimum-threshold-effectiveness measure).

For example, a linear programming method may involve setting constraints on side effects, to generate a feasible region, and selecting a combination of drugs that maximizes the objective function of effectiveness against the biological target, over the feasible region. A further constraint could be that the sum of the proportions of each drug in the multi-drug mixture equals 1. Constraints may also be varied for an individual (or a group of individuals sharing a common characteristic) to accommodate the individual's sensitivity to particular side effects; for example, to accommodate a reduced tolerance for side effects affecting the heart.

As an example of a modeling technique using non-linear methods, neural network models may be employed; this is only one example of a modeling technique, other techniques are readily understood by those skilled in the art. Such models may improve the accuracy of predictions by accounting for non-linear interactive effects that individual drugs of drug combinations may exert upon each other. In a specific example, the assembled data in box 202 may include non-linear interactions between drugs as determined by a variety of sources; these interactions may be described by non-linear functions for an effectiveness measure for a particular compound, side-effect measures for each side-effect, and/or one or more functions representing combinations of effectiveness or side-effect measures. The predictive model of box 203 may include the steps of constructing a neural network model based upon the data of box 202 and any constraints imposed on a combination of effectiveness measures or a combination of side-effect measures as threshold values or optimization constraints. In addition, a neural network model for an objective function is constructed.

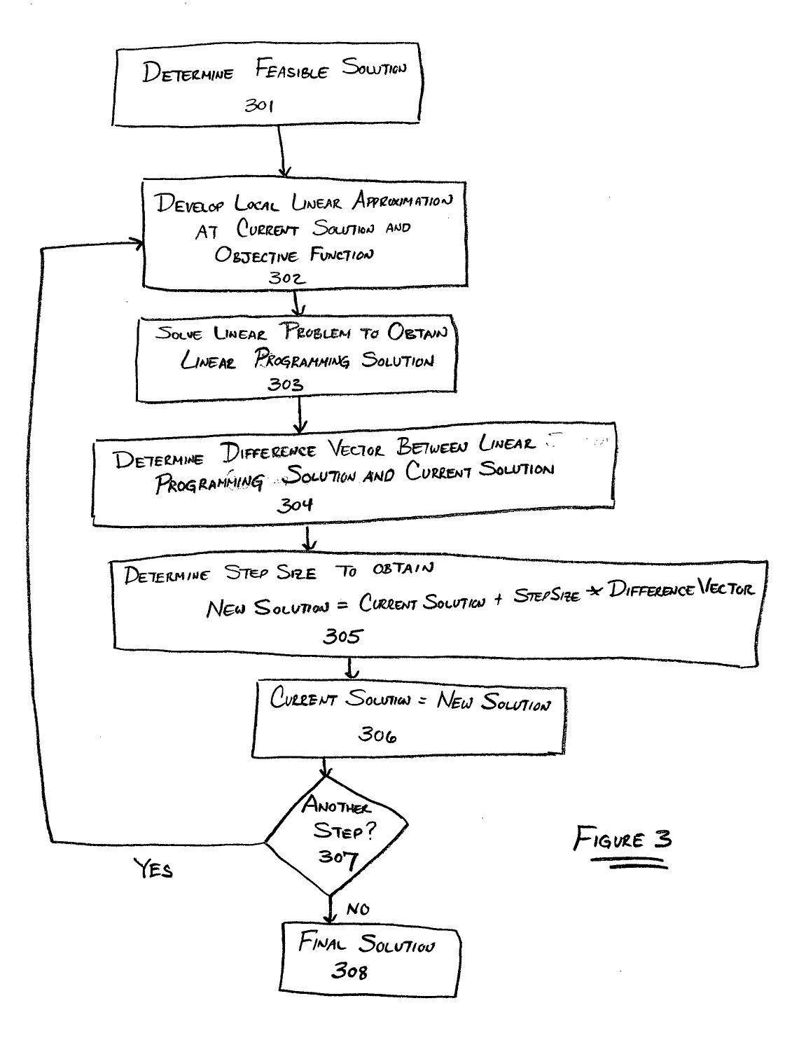

The neural network models may be used to determine a sub-group in the manner diagrammatically depicted in FIG. 3. In box 301, the embodiment of the invention begins by constructing a feasible solution for the models. Such solutions may be constructed using any technique known in the art; for example, standard linear programming techniques may be used to solve a preliminary problem to yield the feasible solution. In box 302, the embodiment utilizes the step of obtaining a local linear approximation to the current solution and objective function; for example, using derivatives or gradients. In box 303, the next step of solving the linear programming problem defined by the model and boxes 301 and 302 is performed. The solution generated by the step of box 303 is a linear programming solution for the local linear approximation. The step of box 304 requires calculating a difference vector, defined by the difference between the linear solution and the current solution. Box 305 is the step of determining a new solution by determining a step size such that the product of the step size and difference vector when summed with the current solution results in a new solution that is consistent with the constraints of the model. The new solution is now designated the current solution in box 306. Then the decision step of 307 is performed in which either the new current solution is utilized iteratively starting in box 302 again, or the solution has reached a condition in which iterations are no longer desired. In the latter case, the new current solution is considered the drug formulation.

Finally, in box 206, the embodiment of FIG. 2 involves administering the drug, which may be performed by administering one dose, or by administering several doses over a time interval. Administering the drug in several doses over a time interval may be advantageous to decrease interactions between compounds, to reduce side effects further, or to increase effectiveness further. The amount of each dose may differ for various administrations. In such an embodiment, the combination of effectiveness measures and combination of side-effect measures for each side effect are a function of time-of-delivery of the sub-group. As well, optimization of the combinations is determined by the constituents of the sub-group, the relative quantities of the constituents, and an administration and dosage schedule. The administration and dosage schedule provides a schedule for when the sub-group should be administered and the dosage of the sub-group at each administration. The relative advantages of such administration of dosages over time may be determined by conducting trials, or as part of the solution to the corresponding optimization problems.

When a drug therapy method includes administering several doses of drugs over time, different combinations of drugs may be used in different separately administered doses. Such an embodiment may be particularly advantageous, for example, when two compounds of a sub-group interfere with each other to produce negative results in a patient. In such a case, each of the interfering drugs may be used in different separately administered doses. In addition, delivery of different combinations of drugs in different separately administered doses may be useful in optimizing drug effectiveness with respect to the patient's system's response to the drug therapy over the time period of administration.

Thus in an embodiment of the invention, a method of providing treatment to a patient, or a group of patients with a similar medical condition, may optimize a combination of effectiveness measures and one or more combinations of side-effect measures for a plurality of compounds. The optimization is performed through the selection of two or more sub-groups of compounds, including the relative quantity of each compound in each sub-group, and the generation of an administration and dosage schedule. Each sub-group represents a separate drug formulation. The administration and dosage schedule provides a schedule of when each drug formulation should be administered and the dosage of the drug formulation to be taken for a particular administration. Drugs may be formed from each sub-group, and administered according to the administration and dosage schedule. The method may also account for one or more characteristics of a patient, or group of patients, as described earlier.

The combination drug may also be used as part of a mixture that includes a partial or fully effective dosage of another drug, that is capable of being effective against the biological target, and of having tolerable side-effects, when used individually against the biological target.

In another embodiment of the invention, the disclosed methods may be used to select promising compounds that may be combined to create new drug formulations. A drug discovery method may begin by selecting a combination of compounds for drug testing from a set of compounds. Tests may include, for example, assays, testing on animals or humans, or other types of laboratory tests. Tests may provide data to describe how a combination may affect a biological target, or provide data to describe how the combination may cause a side effect in a subject who is administered the combination. Tests may include a variety of types of tests, wherein not all compounds are tested or subjected to the same tests. Next, an effectiveness measure and side-effect measure is determined for each compound in the set. Some of the effectiveness measures are derived, in part, from the data collected during the testing. Alternatively, one or more side-effect measures may be determined from the data collected during the testing. An optimization of a combination of effectiveness measures and at least one combination of side-effect measures is performed to determine a sub-group of compounds, and the relative quantity of compounds in the sub-group. Finally, a set of promising compounds for drug testing may be selected by using the results derived from determining the sub-group. The promising compounds may be determined by selecting the candidates such that each candidate compound has at least one similar characteristic to at least one compound in the sub-group. A similar characteristic of two compounds may be defined by physical, chemical, structural, or effective similarities between the compounds. Some examples include a similarity in chemical or molecular structure between the compounds, or the tendency to produce a similar effect when the two compounds are exposed to a particular environment. Such an embodiment may help reduce the cost and effort required in developing new drugs by reducing the set of candidate compounds to those more likely to show a desired activity when combined in a particular manner.

In alternative embodiments, the disclosed methods for creating drug formulations or developing treatments for patients may be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over a network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

Although various exemplary embodiments of the invention have been disclosed, it should be apparent to those skilled in the art that various changes and modifications can be made which will achieve some of the advantages of the invention without departing from the true scope of the invention.

1. A method of creating a drug formulation for producing a desired effect on a biological target, the method comprising:
providing an effectiveness measure for each compound of a plurality of compounds, wherein the effectiveness measure is a measure of predicted effectiveness of the compound in producing the desired effect on the biological target;
providing a side-effect measure for each compound of the plurality of-compounds for each of one or more side effects, wherein the side-effect measure is a measure of predicted risk of a side effect;
performing an optimization of a combination of effectiveness measures and at least one combination of side-effect measures to determine a sub-group of compounds, and a quantity of each compound relative to all other compounds in the sub-group, for the drug formulation; and
making the drug formulation from the sub-group of compounds.

2. A method according to claim 1, wherein performing the optimization includes, for each side effect having a maximum-threshold-side-effect measure, maintaining the combination of side-effect measures below the maximum-threshold-side-effect measure for the side effect.

3. A method according to claim 1, wherein performing the optimization includes limiting the sub-group to compounds having an effectiveness measure above a minimum-threshold-effectiveness-measure.

4. A method according to claim 1, wherein performing the optimization further includes
forming a sub-group from all compounds in the plurality of compounds having an effectiveness measure above a minimum-threshold-effectiveness-measure; and
removing compounds iteratively from the sub-group for each combination of side-effect measures that exceeds the maximum-threshold-side-effect measure, in sequence according to the side-effect measure for each compound beginning with the compound having a measure indicating greatest risk of side effect until the combination of side-effect measures is substantially below the maximum-threshold-side-effect measure.

5. A method according to claim 1, wherein performing the optimization comprises using linear programming.

6. A method according to claim 1, wherein performing the optimization comprises using non-linear programming.

7. A method according to claim 1, wherein the effectiveness measures reflect expected probability of effectiveness.

8. A method according to claim 1, wherein at least one side-effect measure reflects expected probability of occurrence of the side effect.

9. A method according to claim 1, wherein at least one of the measures for one of the compounds is a function of quantity of the compound.

10. A method according to claim 1, wherein the combination of effectiveness measures is derived from a function that depends in part upon an interactive effect between at least two compounds in the sub-group.

11. A method according to claim 10, wherein the function depends in part upon amounts of the at least two compounds of the sub-group.

12. A method according to claim 1, wherein the effectiveness measures are adjusted to reflect predicted effectiveness upon a patient with a particular characteristic.

13. A method according to claim 1, wherein at least one of the side-effect measures is adjusted to reflect predicted risk of side effect in a patient with a particular characteristic.

14. A method according to claim 12, wherein the characteristic is derived from a laboratory test performed on the patient.

15. A method according to claim 12, wherein the characteristic is derived from genetic information from the patient.

16. A method according to claim 12, wherein the characteristic is derived from information from a gene chip array.

17. A method according to claim 1, further comprising setting a feasible region for each combination of side-effect measures, and wherein performing the optimization includes maximizing the combination of effectiveness measures while maintaining each combination of side-effect measures within its respective feasible region.

18. A method according to claim 1, wherein at least one of the combinations of measures is a function of time-of-delivery of the drug formulation to a patient, and the step of performing the optimization includes performing the optimization to determine an administration and dosage schedule for the drug formulation.

19. A method of optimally selecting a set of candidate compounds for discovering a drug formulation that produces a desired effect on a biological target comprising:
testing at least one combination of compounds on the biological target, each combination including at least two compounds from a plurality of compounds;
providing an effectiveness measure for each compound of the plurality of compounds, wherein the effectiveness measure is a measure of predicted effectiveness of the compound in producing the desired effect on the biological target, and is derived in part from the testing;
providing a side-effect measure for each compound of the plurality of compounds for each of one or more side effects, wherein the side-effect measure is a measure of predicted risk of a side effect;
performing an optimization of a combination of effectiveness measures and at least one combination of side-effect measures to determine a sub-group of compounds, and a quantity of each compound relative to all other compounds in the sub-group; and
selecting the set of candidate compounds, wherein each candidate compound has at least one similar characteristic to at least one compound in the sub-group.

20. A method according to claim 19, wherein at least one side-effect measure is derived from the testing.

21. A method of treating a patient comprising:
providing an effectiveness measure for each compound of a plurality of compounds, wherein the effectiveness measure is a measure of predicted effectiveness of the compound in producing a desired effect on a biological target;
providing a side-effect measure for each compound of the plurality of compounds for each of one or more side effects, wherein the side-effect measure is a measure of predicted risk of a side effect;
performing an optimization of a combination of effectiveness measures and at least one combination of side-effect measures to determine a sub-group of compounds, and a quantity of each compound relative to all other compounds in the sub-group, for a drug formulation;
making the drug formulation from the sub-group of compounds; and
administering the drug formulation to the patient.

22. A method according to claim 21, wherein at least one of the combinations of measures is a function of time-of-delivery of the drug formulation to the patient; and performing the optimization includes performing the optimization to determine an administration and dosage schedule; and administering the drug formulation includes administering the drug formulation according to the administration and dosage schedule.

23. A method according to claim 21, wherein the effectiveness measures reflect predicted effectiveness upon the patient with a particular characteristic.

24. A method according to claim 21, wherein at least one of the side-effect measures reflect predicted risk of side effect in the patient with a particular characteristic.

25. A method of treating a patient comprising:
providing an effectiveness measure for each compound of a plurality of compounds, wherein the effectiveness measure is a measure of predicted effectiveness of the compound in producing a desired effect on a biological target;
providing a side-effect measure for each compound of the plurality of compounds for each of one or more side effects, wherein the side-effect measure is a measure of predicted risk of a side effect;
performing an optimization of a combination of effectiveness measures and at least one combination of side-effect measures to determine at least two sub-groups of compounds, and a quantity of each compound relative to all other compounds in a respective sub-group of the compound, and an administration and dosage schedule, wherein each sub-group is associated with one drug formulation, and at least one of the combinations of measures is a function of time-of-delivery of at least one sub-group;
making the drug formulations from the sub-groups of compounds; and
administering each drug formulation to the patient according to the administration and dosage schedule.

26. A method according to claim 25, wherein the effectiveness measures reflect predicted effectiveness upon the patient with a particular characteristic.

27. A method according to claim 25, wherein at least one of the side-effect measures reflect risk of side effect in the patient with a particular characteristic.

28. A drug formulation for producing a desired effect on a biological target made in accordance with claim 1.

29. A drug formulation for producing a desired effect on a biological target comprising a sub-group using compounds in relative quantities in accordance with claim 1.

30. A computer-program product for use on a computer system to identify a drug formulation for producing a desired effect on a biological target, the computer readable program code comprising:
an input module for collecting data on a plurality of compounds and the biological target;
program code to optimize of a combination of effectiveness measures and at least one combination of side-effect measures by computing a sub-group of compounds, and a quantity of each compound relative to all other compounds in the sub-group, for the drug formulation, wherein each effectiveness measure is a measure of predicted effectiveness of the compound in producing the desired effect on the biological target, and each side-effect measure is a measure of predicted risk of a side effect; and
an output module for providing an output including the sub-group of compounds, and relative quantity of each compound in the sub-group.

31. A computer-program product in accordance with claim 30, further comprising program code to compute an effectiveness measure for each compound of the plurality of compounds, wherein the effectiveness measure is a measure of predicted effectiveness of the compound in producing the desired effect on the biological target.

32. A computer-program product in accordance with claim 30, further comprising program code to compute a side-effect measure for each compound of the plurality of compounds for each of one or more side effects, wherein the side-effect measure is a measure of predicted risk of a side effect.

33. A computer-program product in accordance with claim 30, wherein the input module receives a maximum-threshold-side-effect measure for at least one side effect, and the program code to optimize includes program code to optimize such that the set-side-effect measure is below the maximum-threshold-side-effect measure for each of the at least one side effect.

34. A computer-program product in accordance with claim 30, wherein the program code to optimize utilizes linear programming.

35. A computer-program product in accordance with claim 30, wherein the program code to optimize utilizes non-linear programming.

36. A computer-program product in accordance with claim 31 for identifying a drug treatment program using a drug formulation to produce a desired effect on a biological target, wherein the input module also collects data on the patient.

37. A computer-program product in accordance with claim 36, wherein at least one of the combinations of measures is a function of time of delivery of the drug formulation, the program code to optimize includes program code optimize by computing an administration and dosage schedule, and the output module also provides an output of the administration and dosage schedule.

38. A method according to claim 13, wherein the characteristic is derived from a laboratory test performed on the patient.

39. A method according to claim 13, wherein the characteristic is derived from genetic information from the patient.

40. A method according to claim 13, wherein the characteristic is derived from information from a gene chip array.
